(19) United States
(12) Patent Application Publication
(10) Pub. No.: US 2020/0297603 A1
(43) Pub. Date: Sep. 24, 2020

(54) HAIRSTYLING CLAY WITH NON-GREASY SHINE

(71) Applicant: Henkel AG & Co. KGaA, Duesseldorf (DE)

(72) Inventors: NORA KOOPMANN, Hamburg (DE); SANDRA BRANDT, Pinneberg (DE); ARNE JUNGE, Hamburg (DE)

(73) Assignee: Henkel AG & Co. KGaA, Duesseldorf (DE)

(21) Appl. No.: 16/822,447

(22) Filed: Mar. 18, 2020

(30) Foreign Application Priority Data
Mar. 19, 2019 (DE) 10 2019 203 710.0

(51) Int. Cl. A61K 8/37; A61K 8/25; A61K 8/96; A61K 8/92; A61Q 5/06

(52) U.S. Cl. CPC A61K 8/375; A61K 8/25; A61Q 5/06; A61K 8/92; A61K 8/965

(57) ABSTRACT

Cosmetic products which permanently and with a high degree of retention temporarily reshape the treated fibers, giving them a matte and dry, but not greasy appearance, are provided. In one example, the cosmetic product includes, in each case based on the weight of the cosmetic product,
a) from 0 to about 30% by weight of bis-diglyceryl polyacyladipate-1,
b) from 0 to about 30% by weight of bis-diglyceryl polyacyladipate-2,
   wherein the sum of the ingredients a) and b) is >0% by weight,
c) from about 1 to about 35% by weight of diatomaceous earth,
d) from about 0.01 to about 5% by weight of silica,
e) from about 15 to about 60% by weight of water.

### CROSS-REFERENCE TO RELATED APPLICATION

This application claims priority to German Patent Application No. 10 2019 203 710.0, filed Mar. 19, 2019, which is incorporated herein by reference in its entirety.

### TECHNICAL FIELD

The present disclosure concerns cosmetic products for the temporary reshaping of keratinous fibers in the form of so-called “styling clays”. The present disclosure also concerns a method using a cosmetic product.

### BACKGROUND

Cosmetic products that serve to permanently or temporarily shape the hair (styling) play an important role in cosmetics. Therefore, numerous cosmetic products are known in the state of the art, such as hair sprays, hair waxes, hair gels, hair foams, pomades, hair waxes, hair lotions, etc. So-called “styling clays” are a relatively new class of styling products. These products have a consistency from very thick to very creamy. Common to all styling clays is that they contain clay. The clay makes the hair feel “thicker” by giving the individual hair strands body, thickness and structure. Styling clays are particularly suitable for hairstyles that need a lot of volume (for example, “out of bed” look). Besides the clay, styling clays usually contain Vaseline (INCI: petrolatum). Vaseline is an ointment-like mixture of solid and liquid hydrocarbons from petroleum with a melting range of from about 38 to about 58° C. In styling clays, Vaseline is used in particular to protect the hair from humidity and to support malleability. However, Vaseline often leads to a greasy/oily shine of the treated hair. There is also a trend to replace petroleum-based raw materials with renewable and/or natural raw materials. The task underlying the present disclosure is to provide a cosmetic product containing clay which is suitable for the temporary reshaping of keratinous fibers, in which the use of petroleum-based raw materials is significantly reduced or in which the use of petroleum-based raw materials is dispensed with while maintaining the styling properties at a high level. In particular, the hair treated with this product should be given a medium to high stability without the hair having a greasy/oily shine.

### BRIEF SUMMARY

The task underlying the present disclosure is addressed by a cosmetic product for the temporary reshaping of keratinous fibers, containing—in each case based on its weight—
a) from 0 to about 30% by weight of bis-diglyceryl polyacyladipate-1,
b) from 0 to about 30% by weight of bis-diglyceryl polyacyladipate-2,
   with the proviso that the sum of the ingredients a) and b) is >0% by weight,
c) from about 1 to about 35% by weight of diatomaceous earth,
d) from about 0.01 to about 5% by weight of silica,
e) from about 15 to about 60% by weight of water.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has been found that the cosmetic product as contemplated herein is capable of permanently and temporarily reshaping the treated fibers with a high degree of retention. The keratinous fibers are given a matte and dry, but not greasy appearance.

In the context of the present disclosure, keratinous fibers are furs, wool, feathers and hair, in particular human hair. The cosmetic product contains as component a) and/or b) from 0 to about 5% by weight of bis-diglyceryl polyacyladipate-1 and/or from 0 to about 5% by weight of bis-diglyceryl polyacyladipate-2, with the proviso that the sum of the ingredients a) and b) is >0% by weight.

Agents as contemplated herein thus contain
>from 0 to about 30% by weight of bis-diglyceryl polyacyladipate-1 and no bis-diglyceryl polyacyladipate-2 or
>from 0 to about 30% by weight of bis-diglyceryl polyacyladipate-2 and no bis-diglyceryl polyacyladipate-1 or
>from 0 to about 60% by weight of a mixture of bis-diglyceryl polyacyladipate-1 and bis-diglyceryl polyacyladipate-2.

The content of bis-diglyceryl polyacyladipate-1 and/or bis-diglyceryl polyacyladipate-2 in the products as contemplated herein leads to a beautiful clay-like consistency of the product. During application, a strong stability with a very strong texture is achieved. Products without bis-diglyceryl polyacyladipates have a rather creamy consistency which results in a much worse stability of the hairstyle.

Irrespective of whether the means as contemplated herein contain only bis-diglyceryl polyacyladipate-1 (and no bis-diglyceryl polyacyladipate-2) or only bis-diglyceryl polyacyladipate-2 (and no bis-diglyceryl polyacyladipate-1) or both bis-diglyceryl polyacyladipate-1 and bis-diglyceryl polyacyladipate-2, the ingredients a) and/or b) are preferably used within a narrower total quantity range. Cosmetic products preferred as contemplated herein contain a total amount of bis-diglyceryl polyacyladipate-1 and bis-diglyceryl polyacyladipate-2 of from about 2.5 to about 28% by weight, preferably from about 5 to about 26% by weight, more preferably from about 7.5 to about 24% by weight, still more preferably from about 10 to about 22% by weight, still more preferably from about 12.5 to about 21% by weight and in particular from about 15 to about 20% by weight, in each case based on the weight of the entire product.

The use of bis-diglyceryl polyacyladipate-2 is slightly preferred for production reasons, so that particularly preferred cosmetic products contain from about 2.5 to about 28% by weight, preferably from about 5 to about 26% by weight, more preferably from about 7.5 to about 24% by weight, still more preferably from about 10 to about 22% by weight, still more preferably from about 12.5 to about 21% by weight and in particular from about 15 to about 20% by weight of bis-diglyceryl polyacyladipate-2.

As further ingredients, the cosmetic product as contemplated herein contains from about 1 to about 35% by weight of diatomaceous earth; diatomaceous earth, also known as diatomite, diatom earth, also called kieselguhr, rock milk, diatom clay, diatomite, infusorial earth, siliceous earth, novaculite, tripolite, D.E., Celite, is a whitish, powdery substance which consists mainly of the shells of fossil diatoms. The use of diatomaceous earth results in a hairstyle with an excellent texture, which, compared to styling clays based on clay minerals, appears much less greasy and shows the desired “clay-look” even more clearly.

Diatomaceous earth is preferably used within narrower ranges. Cosmetic products as contemplated herein are exemplified here in that they contain diatomaceous earth from about 1.25 to about 30% by weight, preferably from about 1.5 to about 25% by weight, more preferably from about 1.75 to about 20% by weight, still more preferably from about 2 to about 15% by weight, still more preferably from about 2.25 to about 10% by weight and in particular and in particular from about 2.5 to about 5% by weight.

As further ingredients, the cosmetic product as contemplated herein contains from about 0.01 to about 5% by weight of silica. This ingredient also contributes to a significantly less “greasy” appearance of the hairstyle with cosmetic products according to any one of claims 1 to 3, contains from about 0.05 to about 4% by weight, preferably from about 0.1 to about 3.5% by weight, more preferably from about 0.15 to about 3% by weight and more preferably from about 0.2 to about 2% by weight of silica.

The products as contemplated herein contain from about 15 to about 60% by weight of water. For reasons of applicability and stability of the later hairstyle, it is also advantageous here to use water within narrower ranges. Particularly preferred cosmetic products contain from about 17.5 to about 57.5% by weight, preferably from about 20 to about 55% by weight, more preferably from about 22.5 to about 52.5% by weight, still more preferably from about 25 to about 50% by weight, still more preferably from about 27.5 to about 47.5% by weight and in particular from about 30 to about 45% by weight of water.

The products as contemplated herein may contain further ingredients. Oils in particular, and among these preferably natural oils, have proved to be effective as nourishing active ingredients. Preferred cosmetic products as contemplated herein contain from about 2.5 to about 20% by weight, preferably from about 5 to about 17.5% by weight, more preferably from about 7.5 to about 15% by weight and in particular from about 10 to about 12.5% by weight of natural oil(s).

As natural oils, vegetable triglycerides and mixtures of vegetable triglycerides can be used. Preferred natural vegetable oils are coconut oil, (sweet) almond oil, walnut oil, peach kernel oil, apricot kernel oil, avocado oil, tea tree oil, soybean oil, sesame oil, sunflower oil, tsubaki oil, evening primrose oil, rice bran oil, palm kernel oil, mango seed oil, cuckoo flower oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil and ricinus oil. Furthermore, natural carboxylic acid esters such as myristic acid isopropyl ester can be used as natural oils. Myristic acid isopropyl ester (INCI. isopropyl myristate) is, for example, naturally present in butter, palm kernel oil and coconut fat. Animal triglycerides and mixtures of animal triglycerides can also be used as natural oils. A suitable natural animal oil is, for example, mink oil.

In addition to the natural oil(s), the inventive step may also contain natural waxes and/or natural fats.

As natural waxes, vegetable waxes such as candelilla wax, carnauba wax, (partially) hydrogenated ricinus oil, esparto grass wax, Japan wax, cork wax, sugar cane wax, ouricuri wax, montan wax, sunflower wax, cotton wax, rice bran wax, flax wax, peat wax, rose wax, jasmine wax, Cuba palm wax, guaruma wax, shea butter (INCI: butyrospermum parkii (shea) butter), fruit waxes and animal waxes, such as beeswax and other insect waxes, spermaceti, shellac wax, wool wax and rump fat may be used. It may be advantageous to use hydrogenated or hardened waxes.

As natural fats, vegetable fats such as cocoa butter, coconut butter, palm fat, palm kernel fat, bacuri butter, baobab seed oil and/or baobab tree oil can be used. Suitable animal fats include lard and/or beef tallow may be used. In a particularly preferred version, the at least three natural fat compounds comprise a combination of ricinus oil (INCI: Ricinus communis (castor) seed oil), hydrogenated ricinus oil (INCI: hydrogenated castor oil) and carnauba wax (INCI: copernicia cerifera (carnauba) wax).

The cosmetic product preferably contains at least one emulsifier. The at least one emulsifier improves the washability of the cosmetic product. Preferably alkoxylated fatty alcohols may be used as emulsifiers. The preferred emulsifiers offer the advantage that they can be obtained from natural, not fully synthetic raw material sources. Accordingly, preferred cosmetic products are exemplified by the fact that at least one emulsifier comprises an alkoxylated fatty alcohol. An “alkoxylated fatty alcohol” is any fatty alcohol with a carbon chain of C5 or more which further comprises at least one alkoxy group. The fatty alcohol can be selected, for example, from C9-C11 fatty alcohols, C12-C13 fatty alcohols, C12-C15 fatty alcohols, C12-C16 fatty alcohols, C14-C15 fatty alcohols, arachidyl alcohol, behenyl alcohol, caprylic alcohol, cetearyl alcohol, cetyl alcohol, coconut alcohol, decyl alcohol, (hydrogenated) tallow alcohol, lauryl alcohol, myristyl alcohol, oleyl alcohol, palm alcohol, palm kernel alcohol, stearyl alcohol and tridecyl alcohol. The alkoxy group may comprise in particular ethoxy and/or propoxy and/or butoxy groups. The alkoxylated fatty alcohol is particularly preferred to an ethoxylated fatty alcohol. The at least one alkoxy group of the “alkoxylated fatty alcohol” may, for example, be derived from an alkoxylation reaction with alkylene oxide, especially ethylene oxide and/or propylene oxide.

It is preferred that the alkoxylated fatty alcohol is selected from the group including Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Laureth-1, Laureth-2, Laureth-3, Laureth-4, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-23, Laureth-25, Laureth-30, Laureth-40, Deceth-3, Deceth-5, Oleth-2, Oleth-3, Oleth-4, Oleth-5, Oleth-6, Oleth-7, Oleth-8, Oleth-9, Oleth-10, Oleth-11, Oleth-12, Oleth-15, Oleth-16, Oleth-20, Oleth-23, Oleth-25, Oleth-30, Oleth-40, Steareth-2, Steareth-4, Steareth-6, Steareth-7, Steareth-10, Steareth-11, Steareth-13, Steareth-14, Steareth-15, Steareth-20, Steareth-21, Steareth-25, Steareth-27, Steareth-30, Steareth-40, Steareth-50, Steareth-100 and mixtures thereof.

The term Ceteareth-2 stands for a C16-C18 fatty alcohol with an average of 2 ethylene oxide units per molecule.

In a particularly preferred version of the cosmetic product the at least one alkoxylated fatty alcohol comprises Oleth-5.

Preferably, phosphate esters, especially liquid phosphate esters, can also be used as emulsifiers. The term “liquid phosphate ester” refers to any phosphate ester that is liquid at about 20° C. Particularly preferred phosphate esters are Trilaureth-4 phosphates (e.g. available as Hostaphat KL 340D or SILAPHOS® TE 340), C8-C10 phosphates (e.g. available as Crodafos 81 OA), PPG-5-Ceteth-10 phosphates (e.g. available as Crodafos C10/5A), Cetoleth-5 phosphates (e.g. available as Crodafos™ CO5A), Deceth-4 phosphates (e.g. available as Crodafos™ D4A), Glycereth-26 phosphates (e.g. available as Crodafo G26A), Oleth-5 phosphates and dioleyl phosphates (e.g. available as Crodafos HCE), potassium C12-13 alkyl phosphates (e.g. available as Crodafos™ 1213K), TEA C12-13 alkyl phosphates (e.g. available as Crodafos™ 1213T), C9-15 alkyl phosphates (e.g. available as Crodafos M915A), Oleth-10 phosphate (e.g. available as Crodafos O10A), DEA Oleth-10 phosphate (e.g. available as Crodafos™ O10D), Oleth-3 phosphate (e.g. available as Crodafos 03A), DEA Oleth-3 phosphates (e.g. available as Crodafos O3D), Trideceth-10 phosphate (e.g. available as Crodafos T10A), Trideceth-5 phosphates (e.g. available as Crodafos T5A) or Trideceth-6 phosphates (e.g. available as Crodafos™ T6A). It is also possible to use mixtures of phosphate esters. Trilaureth-4 phosphate is the most preferred.

In a particularly preferred form of the cosmetic product, the at least one emulsifier comprises Trilaureth-4 phosphate.

In a particularly preferred form of the cosmetic product, the at least one emulsifier comprises an alkoxylated fatty alcohol and a phosphate ester. In a very preferred form of the cosmetic product, the at least one emulsifier comprises an alkoxylated fatty alcohol and Trilaureth-4 phosphate.

Preferred cosmetic products as contemplated herein contain from about 0.5 to about 25% by weight, preferably from about 1 to about 22.5% by weight, more preferably from about 2 to about 20% by weight, still more preferably from about 3 to about 17.5% by weight, still more preferably from about 4 to about 15% by weight and in particular from about 10 to about 13% by weight of at least one emulsifier from the group of the alkoxylated fatty alcohols, phosphate esters and mixtures thereof.

According to the product name “clay”, the products covered by the present disclosure may contain clay mineral(s).

The term clay mineral refers on the one hand to minerals which are predominantly very fine-grained (grain size <2 μm), but on the other hand to the phyllosilicates, which are named after their layer-like crystal structure including silicon and oxygen, as well as hydrogen and usually magnesium and aluminum. The two definitions are not congruent.

It is preferred that the clay mineral is selected from the group including the kaolin group, the smectite group, the illite-mica group, the chlorite group and mixtures thereof.

It is highly preferred that the clay mineral is a sheet silicate selected from the group including kaolinite, halloysite, dickite, nakrite, dioctahedral vermiculite, dioctahedral illite, and mixtures thereof, dioctahedral smectite, montmorillonite, beidellite, nontronite, volkonskoite, nakrite, trioctahedral vermiculite, trioctahedral smectite, saponite, hectorite, sauconite and mixtures thereof. In a particularly preferred form, this comprises at least one clay mineral kaolinite and/or montmorillonite.

Clay minerals may be added to the product in the form of clays. There are different clays which are exemplified by a different composition of clay minerals and possibly sheet silicates. Alumina is available in white, pink, red-yellow, brown and green. The color of green alumina is due to copper compounds. Iron is the coloring element in pink, red and brown alumina. White clay, also known as kaolin, is a fine, iron-free, white clay containing kaolinite as its main component. Halloysite, dickite and nakrite also occur.

It is particularly preferred that the cosmetic product contains at least one clay mineral in the form of white clay (INCI: kaolin). Accordingly, it is preferred that the at least one clay mineral is selected from the group including kaolinite, halloysite, dickite, nitrite and mixtures thereof. In an especially preferred form, the at least one clay mineral comprises kaolinite, halloysite, dickite and nakrite.

It may also be preferred to add at least one clay mineral in the form of bentonite. Bentonite is an alumina. Bentonite is a mixture of different clay minerals and contains montmorillonite as the most important component (from about 60% to about 80%).

It is also particularly preferred that at least one clay mineral contains bentonite.

With regard to the appearance of the hairstyle produced with the employing the present disclosure, in particular its visual “greasiness”, it is preferable to dispense with the use of clay minerals and in particular kaolin as far as possible. Cosmetic products as contemplated herein, which—based on their weight—contain less than about 1% by weight, preferably less than about 0.5% by weight, more preferably less than about 0.25% by weight, still more preferably less than about 0.1% by weight and still more preferably less than about 0.01% by weight of clay mineral(s), whereby most preferably products are free of clay minerals, are as contemplated herein preferred.

The applicability of the cosmetic product in the hair and the hair feel (touch) after application of the cosmetic product can be further increased by using a fatty acid. Preferred cosmetic products therefore also contain at least one C8 to C24 fatty acid, preferably C12 to C22 fatty acid. Especially preferred cosmetic products contain palmitic acid and/or stearic acid. Relative to the total weight of the cosmetic product, in a preferred embodiment of the present disclosure, from about 1 to about 10% by weight, more preferably from about 4 to about 8% by weight, in each case relative to the total weight of the cosmetic product, of at least one C8- to C24-fatty acid are also contained in the cosmetic product.

In further preferred embodiments of the present disclosure, the cosmetic product also contains, if appropriate, further auxiliary substances, such as preservatives, perfume and/or dyes.

The cosmetic products are preferably in paste form. As containers for the cosmetic product for the temporary reshaping of keratinous fibers, pots are particularly suitable.

A further subject-matter of the present disclosure is the use of the cosmetic product as contemplated herein for the temporary reshaping and/or shape-fixing of keratinous fibers, in particular human hair.

A further subject-matter of the present disclosure is a process for temporarily reshaping keratinous fibers, in particular human hair, in which the cosmetic product as contemplated herein is applied to the keratinous fibers.

### Examples

The following styling clays (figures in % by weight) were produced:

| | E1 | E2 | E3 | E4 | E5 |
|---|---|---|---|---|---|
| Bis-diglyceryl polyacyladipate-2 | 20.0 | 21.0 | 22.0 | 23.0 | 24.0 |
| Diatomaceous earth | 3.0 | 2.5 | 3.0 | 2.5 | 3.0 |
| Silica | 1.5 | 2.0 | 2.5 | 2.0 | 1.5 |
| Hydrogenated vegetable oil | 10.0 | 11.0 | 12.0 | 13.0 | 14.0 |
| Bees wax | 7.0 | 7.0 | 7.0 | 7.0 | 7.0 |
| PEG-7 glyceryl cocoate | 6.0 | 6.0 | 6.0 | 6.0 | 6.0 |
| Ceteareth-20 | 5.0 | 5.0 | 5.0 | 5.0 | 5.0 |
| Emulsifier (cetearyl alcohol | 5.0 | 5.0 | 5.0 | 5.0 | 5.0 |
| and cetearyl glucoside) | | | | | |
| Isopropyl myristate | 2.0 | 2.0 | 2.0 | 2.0 | 2.0 |
| Preservative (phenoxyethanol/ | 1.0 | 1.0 | 1.0 | 1.0 | 1.0 |
| ethylhexylglycerin) | | | | | |
| Perfume | 1.5 | 2.0 | 2.5 | 3.0 | 3.5 |
| Water | ad 100% | ad 100% | ad 100% | ad 100% | ad 100% |
| | E6 | E7 | E8 | E9 | E10 |
| Bis-diglyceryl polyacyladipate-2 | 20.0 | 19.0 | 18.0 | 17.0 | 16.0 |
| Diatomaceous earth | 3.0 | 3.5 | 4.0 | 4.5 | 5.0 |
| Silica | 1.5 | 2.0 | 2.5 | 2.0 | 1.5 |
| Hydrogenated vegetable oil | 10.0 | 10.0 | 10.0 | 10.0 | 10.0 |
| Olive oil | 3.0 | 3.0 | 3.0 | 3.0 | 3.0 |
| Bees wax | 7.0 | 7.0 | 7.0 | 7.0 | 7.0 |
| PEG-7 glyceryl cocoate | 6.0 | 6.0 | 6.0 | 6.0 | 6.0 |
| Ceteareth-20 | 5.0 | 5.0 | 5.0 | 5.0 | 5.0 |
| Emulsifier (cetearyl alcohol | 5.0 | 5.0 | 5.0 | 5.0 | 5.0 |
| and cetearyl glucoside) | | | | | |
| Isopropyl myristate | 2.0 | 2.0 | 2.0 | 2.0 | 2.0 |
| Preservative (phenoxyethanol/ | 1.0 | 1.0 | 1.0 | 1.0 | 1.0 |
| ethylhexylglycerin) | | | | | |
| Perfume | 3.5 | 3.5 | 3.5 | 3.5 | 3.5 |
| Water | ad 100% | ad 100% | ad 100% | ad 100% | ad 100% |

The formula E6 as contemplated herein was tested against the two comparison formulas V1 and V2 in the hair studio. V1 contained no silica, in V2 the use of bis-diglyceryl polyacyladipate-2 was dispensed with.

In the hair studio, 0.5 g of each product was applied to the heads of 30 test persons and the test persons were asked to give their evaluation of E6, V1 and V2 with regard to the criteria listed below on a scale of 1 (poor) to 6 (excellent).

The formulas are shown in the table below:

| | E6 | V1 | V2 |
|---|---|---|---|
| Bis-diglyceryl polyacyladipate-2 | 20.0 | 20.0 | — |
| Diatomaceous earth | 3.0 | 3.0 | 3.0 |
| Silica | 1.5 | — | 1.5 |
| Hydrogenated vegetable oil | 10.0 | 10.0 | 10.0 |
| Olive oil | 3.0 | 3.0 | 3.0 |
| Bees wax | 7.0 | 7.0 | 7.0 |
| PEG-7 glyceryl cocoate | 6.0 | 6.0 | 6.0 |
| Ceteareth-20 | 5.0 | 5.0 | 5.0 |
| Emulsifier (cetearyl alcohol | 5.0 | 5.0 | 5.0 |
| and cetearyl glucoside) | | | |
| Isopropyl myristate | 2.0 | 2.0 | 2.0 |
| Preservative (phenoxyethanol/ | 1.0 | 1.0 | 1.0 |
| ethylhexylglycerin) | | | |
| Perfume | 3.5 | 3.5 | 3.5 |
| Water | ad 100% | ad 100% | ad 100% |

The evaluation regarding the 12 criteria was as follows (arithmetic mean):

| | E6 | V1 | V2 |
|---|---|---|---|
| Texturizing on dry hair | 5.33 | 4.00 | 4.00 |
| Consistency | 5.00 | 4.67 | 3.67 |
| Ease of styling | 5.00 | 4.67 | 4.33 |
| Feel of dry hair | 5.00 | 3.67 | 4.33 |
| Look of dry hair | 5.00 | 3.67 | 4.33 |
| Control | 5.00 | 4.00 | 4.00 |
| Hair structure | 5.33 | 4.33 | 4.00 |
| Residues | 5.00 | 5.00 | 4.67 |
| Shine | 4.67 | 4.33 | 4.67 |
| Remouldability | 5.00 | 5.00 | 4.67 |
| Rinseability | 5.00 | 5.00 | 4.67 |
| Non greasy | 5.00 | 4.00 | 4.00 |
| Mean value | 5.03 | 4.36 | 4.28 |

The results show that the product as contemplated herein is consistently rated better than the comparison products.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic product for the temporary reshaping of keratinous fibers, comprising, based on the weight of the cosmetic product,
a) from 0 to about 30% by weight of bis-diglyceryl polyacyladipate-1,
b) from 0 to about 30% by weight of bis-diglyceryl polyacyladipate-2,
wherein the sum of the ingredients a) and b) is greater than 0% by weight,
c) from about 1 to about 35% by weight of diatomaceous earth,
d) from about 0.01 to about 5% by weight of silica,
e) from about 15 to about 60% by weight of water.

2. Cosmetic product according to claim 1, wherein the cosmetic product comprises from about 2.5 to about 28% by weight of bis-diglyceryl polyacyladipate-2.

3. Cosmetic product according to claim 1, wherein the cosmetic product comprises from about 1.25 to about 30% by weight of diatomaceous earth.

4. Cosmetic product according to claim 1, wherein the cosmetic product comprises from about 0.05 to about 4% by weight of silica.

5. Cosmetic product according to claim 1, wherein the cosmetic product comprises from about 17.5 to about 57.5% by weight of water.

6. Cosmetic product according to claim 1, wherein the cosmetic product comprises from about 2.5 to about 20% by weight of natural oil(s).

7. Cosmetic product according to claim 1, wherein the cosmetic product comprises less than about 1% by weight of clay minerals.

8. Cosmetic product according to claim 1, wherein the cosmetic product comprises from about 0.5 to about 25% by weight of at least one emulsifier from the group of alkoxylated fatty alcohols, phosphate esters and mixtures thereof.

9. Method comprising the step of using the cosmetic product according to claim 1 for the temporary reshaping and/or shape-fixing of keratin fibers.

10. Method for the temporary reshaping of keratinous fibers, that the method comprising the step of applying the cosmetic product of claim 1 to the keratinous fibers.

11. Cosmetic product according to claim 1, wherein the cosmetic product comprises from about 5 to about 26% by weight of bis-diglyceryl polyacyladipate-2.

12. Cosmetic product according to claim 1, wherein the cosmetic product comprises from about 10 to about 22% by weight of bis-diglyceryl polyacyladipate-2.

13. Cosmetic product according to claim 1, wherein the cosmetic product comprises from about 15 to about 20% by weight of bis-diglyceryl polyacyladipate-2.

14. Cosmetic product according to claim 1, wherein the cosmetic product comprises from about 2.5 to about 5% by weight of diatomaceous earth.

15. Cosmetic product according to claim 1, wherein the cosmetic product comprises from about 0.2 to about 2% by weight of silica.

16. Cosmetic product according to claim 1, wherein the cosmetic product comprises from about 30 to about 45% by weight of water.

17. Cosmetic product according to claim 1, wherein the cosmetic product comprises from about 10 to about 12.5% by weight of natural oil(s).

18. Cosmetic product according to claim 1, wherein the cosmetic product is free of clay minerals.

19. Cosmetic product according to claim 1, wherein the cosmetic product comprises from about 10 to 13% by weight of at least one emulsifier from the group of alkoxylated fatty alcohols, phosphate esters and mixtures thereof.
